(19) United States
(12) Patent Application Publication
(10) Pub. No.: US 2026/0227393 A1
(43) Pub. Date: Aug. 6, 2026

(54) REAGENT FOR DETECTING ALBUMIN AND METHOD FOR DETECTING ALBUMIN

(71) Applicant: NEC Corporation, Tokyo (JP)

(72) Inventor: Katsumi MAEDA, Tokyo (JP)

(73) Assignee: NEC Corporation, Tokyo (JP)

(21) Appl. No.: 19/432,295

(22) Filed: Dec. 24, 2025

(30) Foreign Application Priority Data
Jan. 15, 2025 (JP) 2025-005165

(51) Int. Cl. G01N 33/543; G01N 21/64; G01N 33/68

(52) U.S. Cl. CPC G01N 33/54386; G01N 21/6428; G01N 33/6893; G01N 2021/6419; G01N 2021/6439; G01N 2333/76

(57) ABSTRACT

To provide a reagent for detecting albumin, a method for detecting albumin in a sample, or the like.

A reagent for detecting albumin, the reagent comprising a europium complex represented by Formula (1):

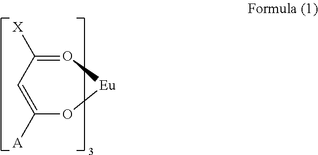

wherein X represents a substituted or unsubstituted aryl group or heterocyclic group, and A represents a substituted or unsubstituted alkyl group.

This application is based upon and claims the benefit of priority from Japanese patent application No. 2025-5165, filed on Jan. 15, 2025, the disclosure of which is incorporated herein in its entirety by reference.

### TECHNICAL FIELD

The present disclosure relates to a reagent for detecting albumin and a method for detecting albumin.

### BACKGROUND ART

Albumin is a protein that has a molecular weight of about 66,000 and accounts for 50 to 65% of the proteins present in serum. Albumin has an action of maintaining the osmotic pressure of blood, and further has a function of binding to and transporting hormones, fatty acids, drug molecules, metal ions, and the like. Albumin is known to be associated with diseases such as kidney disease, cardiovascular disease, and liver disease.

In healthy individuals, albumin is filtered in the kidneys. When albumin is not properly filtered in the kidneys, albumin is excreted in urine. It is known that, for example, in a patient with diabetic nephropathy that is one of complications of diabetes, the amount of albumin contained in urine increases. Therefore, the urinary albumin value is an index for diagnosing diabetic nephropathy.

A urine test paper method and a turbidimetric immunoassay (a TIA method) are known as methods for detecting albumin. A detection method utilizing a reaction of albumin with a fluorescent organic compound has also been reported. For example, Non-patent Literature 1 discloses that 4-[4-(4-dimethylaminophenyl)-5-phenyl-1H-imidazol-2-yl]benzoic acid methyl ester (DAPIM) has a fluorescence property that the fluorescence of DAPIM is increased by specific binding to human serum albumin (HSA), and can be used for the detection of diabetic nephropathy.

### CITATION LIST

Non-patent Literature 1: Nagasaki University Graduate School of Biomedical Sciences, “Development of a novel on-site screening method that enables early detection of diabetic nephropathy—aiming at reduction in the number of dialysis induction in the elderly and reduction in medical cost”, Mitsui Sumitomo Insurance Welfare Foundation, Report of Research Results, Vol. 17, Research Grant 2011 (issued in July 2013), available online (URL: https://www.ms-ins.com/welfare/document/list/list2011.htm)

### SUMMARY

However, the method for detecting albumin in Non-patent Literature 1 has room for improvement from the viewpoint of sensitivity. Therefore, an object of one aspect of the present disclosure is to provide a reagent for detecting albumin, a method for detecting albumin in a sample, and a device for detecting albumin, or the like.

One aspect of the present disclosure relates to a reagent for detecting albumin, the reagent comprising a europium complex represented by the following Formula (1):

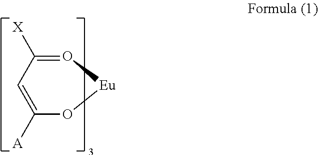

wherein X represents a substituted or unsubstituted aryl group or heterocyclic group, and A represents a substituted or unsubstituted alkyl group.

In addition, one aspect of the present disclosure relates to a method for detecting albumin in a sample using the europium complex represented by Formula (1).

One aspect of the present disclosure can provide a reagent for detecting albumin. In addition, one aspect of the present disclosure can provide a method for detecting albumin in a sample, or the like.

### BRIEF DESCRIPTION OF THE DRAWINGS

FIG. 1 is a diagram showing a fluorescence spectrum curve of a reaction product of albumin and a europium complex according to the present disclosure, and a fluorescence spectrum curve of the europium complex alone;

FIG. 2 is a diagram showing a fluorescence spectrum curve of a reaction product of albumin and a europium complex according to the present disclosure, and a fluorescence spectrum curve of the europium complex alone;

FIG. 3 is a diagram showing a fluorescence spectrum curve of a reaction product of albumin and a europium complex according to the present disclosure, and a fluorescence spectrum curve of the europium complex alone;

FIG. 4 is an example of a photograph in which fluorescence emission of the reaction product of albumin and a europium complex according to the present disclosure has been observed;

FIG. 5 is a diagram showing a fluorescence spectrum curve of a reaction product of albumin and a europium complex according to the present disclosure, and a fluorescence spectrum curve of the europium complex alone;

FIG. 6 is a diagram showing a fluorescence spectrum curve of a reaction product of albumin and a europium complex according to the present disclosure, and a fluorescence spectrum curve of the europium complex alone;

FIG. 7 is an example of a photograph in which fluorescence emission of the reaction product of albumin and a europium complex according to the present disclosure has been observed;

FIG. 8 is a diagram showing a fluorescence spectrum curve of a reaction product of albumin and a europium complex according to the present disclosure, and a fluorescence spectrum curve of the europium complex alone;

FIG. 9 is an example of a photograph in which fluorescence emission of the reaction product of albumin and a europium complex according to the present disclosure has been observed;

FIG. 10 is a diagram showing a fluorescence spectrum curve of a reaction product of albumin and a europium complex according to the present disclosure, and a fluorescence spectrum curve of the europium complex alone;

FIG. 11 is an example of a photograph in which fluorescence emission of the reaction product of albumin and a europium complex according to the present disclosure has been observed;

FIG. 12 is a diagram showing a fluorescence spectrum curve of a reaction product of albumin and a europium complex according to the present disclosure, and a fluorescence spectrum curve of the europium complex alone;

FIG. 13 is an example of a photograph in which fluorescence emission of the reaction product of albumin and a europium complex according to the present disclosure has been observed;

FIG. 14 is a diagram showing a fluorescence spectrum curve of a reaction product of albumin and a europium complex according to the present disclosure, and a fluorescence spectrum curve of the europium complex alone;

FIG. 15 is a diagram showing a fluorescence spectrum curve of a reaction product of albumin and a europium complex according to the present disclosure, and a fluorescence spectrum curve of the europium complex alone;

FIG. 16 is an example of a photograph in which fluorescence emission of the reaction product of albumin and a europium complex according to the present disclosure has been observed;

FIG. 17 is a diagram showing a fluorescence spectrum curve of a reaction product of albumin and a europium complex according to the present disclosure, and a fluorescence spectrum curve of the europium complex alone; and

FIG. 18 is an example of a photograph in which fluorescence emission of the reaction product of albumin and a europium complex according to the present disclosure has been observed.

### EXAMPLE EMBODIMENT

Hereinafter, embodiments for carrying out the present disclosure will be described, but the example embodiments according to the present disclosure are not limited to the following embodiments.

### <Reagent for Detecting Albumin>

In the present disclosure, the term “reagent” is defined as a chemical substance used for detection or quantification of a substance by a chemical method, an experiment of synthesis of a substance, or measurement of physical properties.

In the present disclosure, albumin may be, but not limited to, human serum albumin (HSA), bovine serum albumin (BSA), or the like.

### (Europium Complex)

A reagent for detecting albumin of the present disclosure (also simply referred to as a “reagent”) contains a europium complex represented by the following formula (1) (also simply referred to as an “europium complex” or an “europium complex of the formula (1)”).

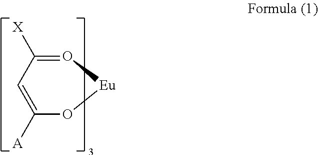

wherein X represents a substituted or unsubstituted aryl group or heterocyclic group, and A represents a substituted or unsubstituted alkyl group.

In the formula (1), X is a substituted or unsubstituted aryl group or heterocyclic group. The aryl group refers to a monovalent aromatic hydrocarbon group. The aryl group is preferably an aryl group having 6 to 18 carbon atoms, and more preferably an aryl group having 6 to 10 carbon atoms. Examples of the aryl group include a phenyl group, a 1-naphthyl group, and a 2-naphthyl group, and a phenyl group and a 2-naphthyl group are preferable.

The heterocyclic group refers to a group obtained by removing one hydrogen atom from a heterocyclic compound containing an oxygen atom, a nitrogen atom, or a sulfur atom as a hetero atom. The heterocyclic group is preferably a heterocyclic group having 2 to 30 carbon atoms, and more preferably a heterocyclic group having 4 to 20 carbon atoms. Examples of the heterocyclic group include a thienyl group, a pyrrolyl group, a furyl group, a pyridyl group, a piperidyl group, a quinolyl group, an isoquinolyl group, a pyrimidinyl group, and a triazinyl group, and a thienyl group, a furyl group, and a pyridyl group are preferable.

The substituent in the substituted aryl group and the substituted heterocyclic group is not particularly limited, and examples thereof include a halogen atom, a cyano group, an alkyl group, an alkoxy group, an alkylcarbonyl group, an aryl group, a heteroaryl group, an aryloxy group, an arylcarbonyl group, an aralkyl group, an amino group, a nitro group, and a hydroxy group, and the substituent is preferably a halogen atom, an alkyl group, or an alkoxy group. The number of substituents is preferably 1 to 3, and more preferably 1.

In the formula (1), X is particularly preferably a 2-thienyl group, a 2-furyl group, a 2-pyridyl group, a phenyl group, a 2-naphthyl group, a 4-hydroxyphenyl group, a 4-methoxyphenyl group, a 2,4-dimethoxyphenyl group, or a 4-fluorophenyl group.

In the formula (1), A is a substituted or unsubstituted alkyl group. The alkyl group is preferably an alkyl group having 1 to 50 carbon atoms, more preferably an alkyl group having 1 to 30 carbon atoms, and still more preferably an alkyl group having 1 to 20 carbon atoms. Examples of the alkyl group include a methyl group, an ethyl group, a n-propyl group, an isopropyl group, a n-butyl group, an isobutyl group, a tert-butyl group, a n-pentyl group, an isoamyl group, a 2-ethylbutyl group, a n-hexyl group, a cyclohexyl group, a n-heptyl group, a cyclohexylmethyl group, a cyclohexylethyl group, a n-octyl group, a 2-ethylhexyl group, a 3-n-propylheptyl group, a n-decyl group, a 3,7-dimethyloctyl group, a 2-ethyloctyl group, a 2-n-hexyl-decyl group, a n-dodecyl group, a tetradecyl group, a hexadecyl group, an octadecyl group, an eicosyl group, a cyclopentyl group, a cyclohexyl group, a cycloheptyl group, and an adamantyl group, and a methyl group is preferable.

The substituent in the substituted alkyl group is not particularly limited, and examples thereof include a halogen atom, a cyano group, an alkoxy group, a carbonyl group, an aryl group, a heteroaryl group, an aryloxy group, an arylcarbonyl group, an aralkyl group, an amino group, a nitro group, and a hydroxy group, and the substituent is preferably a halogen atom, and more preferably a fluorine atom. The number of substituents is preferably 1 to 3, and more preferably 1.

In the formula (1), A is particularly preferably a trifluoromethyl group, a pentafluoroethyl group, or a heptafluoropropyl group.

Examples of the europium complex of the formula (1) include, but are not limited to, the following europium complexes Eu-1 to Eu-6.

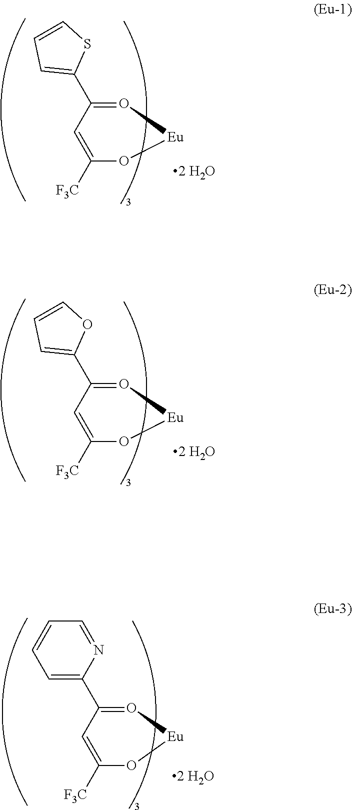

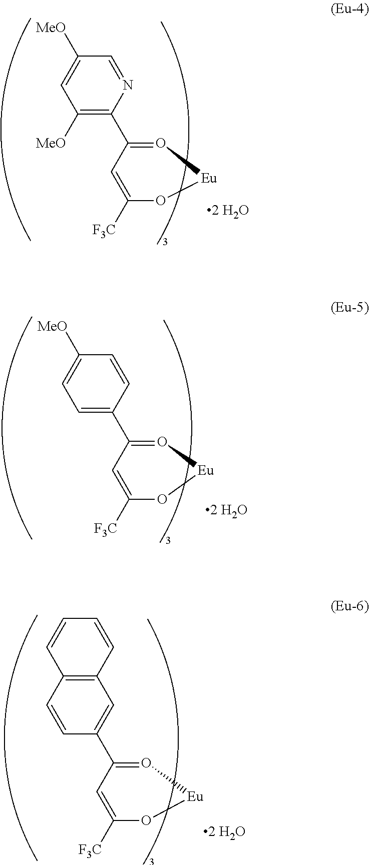

In one aspect, the europium complex Eu-1 (europium complex synthesized using 4,4,4-trifluoro-1-(2-thienyl)-1,3-butanedione as a raw material), the europium complex Eu-5 (europium complex synthesized using 4,4,4-trifluoro-1-(4-dimethoxyphenyl)-1,3-butanedione as a raw material), and the europium complex Eu-6 (europium complex synthesized using 4,4,4-trifluoro-1-(2-naphthyl)-1,3-butanedione as a raw material) are particularly suitable for detecting albumin, but the present disclosure is not limited thereto.

The reagent of the present disclosure may contain one europium complex alone, or may contain two or more europium complexes in combination.

The reagent for detecting albumin of the present disclosure contains the europium complex of the formula (1), and in fluorescence intensity measurement at a specific wavelength, albumin in the sample can be detected because the fluorescence intensity emitted from a mixture of the europium complex and albumin (also referred to as a “reaction product”) is larger than the fluorescence intensity of the europium complex alone.

### (Method for Producing Europium Complex)

The europium complex of the formula (1) can be synthesized, for example, by reacting europium(III) chloride hexahydrate or europium(III) nitrate hexahydrate with a compound represented by the following formula (2):

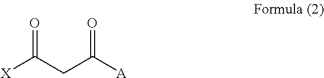

wherein X represents a substituted or unsubstituted aryl group or heterocyclic group, and A represents a substituted or unsubstituted alkyl group.)

For example, in the formula (1), in a case of synthesizing the europium complex Eu-1, where X is a 2-thienyl group and A is a trifluoromethyl group in the formula (1), the desired europium complex Eu-1 can be obtained by reacting 4,4,4-trifluoro-1-(2-thienyl)-1,3-butanedione and europium(III) chloride hexahydrate in the presence of sodium hydroxide.

### (Solvent)

The reagent of the present disclosure may contain a solvent in addition to the europium complex of the formula (1). The solvent is preferably, but not limited to, an organic solvent capable of dissolving the europium complex, and examples of the solvent include dimethyl sulfoxide, methanol, ethanol, N,N-dimethylformamide, tetrahydrofuran, acetone, acetonitrile, and 1,4-dioxane. The solvent may be one kind or a mixture of two or more kinds.

In a case where a solution in which the europium complex of the formula (1) is dissolved in a solvent is used as a reagent, the concentration of the europium complex represented by the formula (1) in the solution (the concentration before mixing with the sample) is not limited, but may be, for example, preferably equal to or more than 0.1 mM, equal to or more than 0.2 mM, equal to or more than 0.5 mM, equal to or more than 1.0 mM, equal to or more than 1.5 mM, or equal to or more than 2.0 mM, and may be preferably equal to or less than 10.0 mM, equal to or less than 8.0 mM, equal to or less than 6.0 mM, or equal to or less than 5.0 mM. In the present specification, the concentration unit “M” means “mol/L”.

### (Glycol-Based Compound, and Glycerol)

The reagent of the present disclosure may further contain a glycol-based compound or glycerol as an additive in addition to the europium complex of the formula (1). The addition of the glycol-based compound or glycerol is preferable because of its ability to facilitate the reaction between the reagent and albumin. Examples of the glycol-based compound include ethylene glycol, diethylene glycol, triethylene glycol, and polyethylene glycol. The amount of the glycol-based compound or glycerol is preferably 0% to 50% by volume with respect to the amount of the solvent.

### <Method for Detecting Albumin in Sample>

In one aspect of the present disclosure, a method for detecting albumin in a sample includes detecting fluorescence emission of a mixture of the europium complex represented by the formula (1) and albumin. In one preferred aspect, the method for detecting albumin in a sample includes: (i) obtaining a mixture of a sample and a reagent containing the europium complex represented by the formula (1) described above (step (i)), (ii) irradiating the mixture with excitation light having a specific wavelength (step (ii)), and (iii) detecting fluorescence emitted from the mixture (step (iii)). The step (i) is preferably carried out in water. The method for detecting albumin of the present disclosure utilizes a phenomenon in which the fluorescence emission increases due to the interaction between the europium complex represented by the formula (1) and albumin that are mixed and brought into contact with each other. While the europium complex alone exhibits little fluorescence emission in an aqueous solution, the mixture (reaction product) obtained by the reaction of the europium complex with albumin newly exhibits fluorescence emission. The albumin in the sample can be detected by utilizing this phenomenon.

A urine test strip method, a turbidimetric immunoassay (TIA method), and the like have been known as a method for detecting albumin. However, although the known urine test strip method is inexpensive and can easily detect albumin, the urine test strip method may not be sensitive enough, and may not detect the histological change of the kidney due to, for example diabetic nephropathy, even when the histological change of the kidney has already progressed. In addition, the method for detecting albumin by the TIA method tends to have high sensitivity, but is expensive and has complicated detection work. Therefore, there has been a demand for development of a reagent for detecting albumin in a sample, in which the reagent can be used simply, is inexpensive, and can detect albumin with high sensitivity even when the concentration of albumin is low.

The method for detecting albumin of the present disclosure is inexpensive, can be carried out easily, and can detect albumin in a sample with high sensitivity even when the concentration of albumin in the sample is low. Therefore, the method for detecting albumin of the present disclosure is also considered to be useful for early detection, prevention, deceleration of the progression, treatment, and the like of diabetic nephropathy or the like in which the amount of albumin in urine increases.

In one aspect of the present disclosure, the sample is preferably, but not limited to, a biological sample, and examples of the sample include blood, plasma, serum, lymph, saliva, sweat, tears, and urine, and in one aspect, the sample is preferably urine. The biological sample is preferably derived from a mammal, but is not limited thereto, and examples of the mammal include human, cow, goat, sheep, pig, monkey, dog, cat, rat, mouse, hamster, and guinea pig, and the biological sample is more preferably human. The sample may be a solid or a solution. The solution may be body fluid, may contain a solvent that is not body fluid, or may be a mixture thereof. The solution may be a collected sample used as it is, or may be a liquid in which the collected sample is diluted with water or the like or concentrated. In one aspect, the solution may be a solution used for sample measurement, a solution used for measurement for calibration, or a standard solution or a calibration solution.

The mixing (contacting) of the sample and the reagent in the step (i) is preferably performed in the presence of albumin under the condition that albumin and the europium complex can react, more preferably performed in a solution, and still more preferably performed in an aqueous solution. In one aspect, a solution containing the europium complex of the formula (1) and the sample (preferably containing water) are preferably mixed, stirred as necessary, and reacted at room temperature (about 5° C. to 40° C., and preferably about 15° C. to 35° C.) for equal to or more than 30 seconds, and preferably equal to or more than 1 minute (for example, 1 to 120 minutes, and preferably 1 to 60 minutes).

The mixing ratio of the reagent containing the europium complex to the sample is not limited, and can be appropriately adjusted depending on factors such as the type of the europium complex to be used, the concentration of albumin in the sample, and the like. In one aspect, for example, the mixing is preferably performed in such a way that the europium complex is preferably equal to or more than 1×10⁻⁶ mol, and more preferably equal to or more than 1×10⁻⁵ mol, based on 1 mg of albumin. In one aspect, when the sample and the reagent are mixed in the step (i), the volume of the sample (aqueous solution) may be, for example, equal to or more than 30 times, equal to or more than 50 times, or equal to or more than 80 times the volume of the solution (reagent) containing the europium complex, and mixing may be performed in such a way that the volume of the sample is equal to or less than 500 times, equal to or less than 300 times, or equal to or less than 200 times the volume of the solution (reagent) containing the europium complex, but not limited thereto. In one aspect, for example, the mixing may be performed in such a way that {the volume of the reagent containing the europium complex (preferably a solution having a concentration of 0.1 mM to 10 mM)}: {the volume of the sample (aqueous solution)} is 1:30 to 1:200, and preferably 1:80 to 1:150.

In one aspect, the reaction of a reagent containing the europium complex of the formula (1) with a sample may be performed in a solid medium containing the reagent. The solid medium may be, for example, but not limited to, paper (for example, filter paper), glass (for example, glass fiber, and porous glass substrate), a resin (for example, polymethyl methacrylate, polyethylene, polypropylene, polyvinyl chloride, polystyrene, nylon resin, polyamide, polycarbonate, polyethylene terephthalate, polybutylene terephthalate, and polyphenylene oxide), or a water soluble polymer (cellulose-based, agarose, starch-based, sodium alginate, acrylic acid-based, acrylamide-based, polyvinyl alcohol, polyethylene oxide, polyvinyl pyrrolidone, and the like).

Examples of the method for detection in a solid medium include a method in which a test strip (for example, filter paper or the like) is impregnated with a solution containing the europium complex of the formula (1) and then dried at 60° C. from room temperature to obtain a test strip containing the europium complex, and the test strip is immersed in an aqueous solution of a sample containing albumin or the aqueous solution is added dropwise to the test strip, and after a reaction occurs, fluorescence emission is detected.

In the step (ii), the mixture obtained in the step (i) is irradiated with excitation light. The wavelength of the excitation light to be emitted is not limited, and is, for example, preferably 200 to 500 nm and more preferably 300 to 400 nm.

In the step (iii), while the europium complex alone exhibits little fluorescence emission, a reaction product of the europium complex and albumin newly exhibits fluorescence emission, and albumin can be detected by utilizing this phenomenon. In one aspect, determining the concentration of albumin by comparing the intensity of the detected fluorescence to a predetermined reference value may also be performed.

The concentration of albumin in the sample may be, for example, equal to or more than 0.0001 mg/mL, equal to or more than 0.0005 mg/mL, equal to or more than 0.001 mg/mL, or equal to or more than 0.01 mg/mL, and may be, for example, equal to or less than 100 mg/mL, equal to or less than 50 mg/mL, equal to or less than 10 mg/mL, equal to or less than 5 mg/mL, or 3 mg/mL. The europium complex in the formula (1) of the present disclosure can detect the presence of albumin in a sample with high sensitivity even when the concentration of albumin in the sample is low (for example, the concentration of albumin may be less than 0.3 mg/mL, equal to or less than 0.1 mg/mL, equal to or less than 0.01 mg/mL, or less than 0.01 mg/mL).

One aspect of the present disclosure relates to a kit for detecting albumin containing the reagent.

### <Device for Detecting Albumin>

One aspect of the present disclosure relates to a device for detecting albumin (also simply referred to as a “detection device” or an “albumin detection device”) including a reagent containing the europium complex represented by the formula (1). In one aspect, an albumin detection device includes an albumin capturing unit having a reagent containing the europium complex represented by the formula (1), and a detection unit for detecting that albumin has been captured in the capturing unit.

### (Capturing Unit)

The capturing unit of the albumin detection device of the present disclosure includes a reagent containing the europium complex of the formula (1). For the reagent, the description of the reagent for detecting albumin described above applies. In one aspect, the reagent contains a solvent (preferably an organic solvent), and is preferably a solution in which the europium complex of the formula (1) is dissolved. In one aspect, the sample and the reagent may be mixed in the capturing unit.

### (Detection Unit)

The detection unit of the albumin detection device is preferably configured to be able to optically detect that albumin has been captured in the capturing unit. The detection unit may be configured as a separate device from the capturing unit rather than as a device in which the capturing unit is integrated with the detection unit. In one aspect, in order to detect the fluorescence emission of a reaction product of the europium complex of the formula (1) and albumin, the optical detection unit includes an excitation light source (light emitting unit) and a detection element (light receiving unit of fluorescence), and can detect and/or measure the concentration of albumin based on the observed change in fluorescence intensity.

In one aspect, in order to detect the fluorescence emission of the reaction product of the europium complex of the formula (1) and albumin, the detection and/or concentration measurement of albumin can be achieved by capturing an image of the fluorescence emission observed when induced by the excitation light source (light emitting unit), and comparing the light-emitting state of the obtained image with a predetermined reference value.

In one aspect, the detection unit may include a computer that executes a computer program for processing the detection and/or the measurement of the concentration of albumin. Such a computer program may be, for example, a computer program that causes a computer to execute the stages of: (i) receiving a signal from an optical detection element; (ii) analyzing the received signal to determine the presence or absence and/or the concentration of albumin; and (iii) outputting an analysis result.

In one aspect of the present disclosure, the analysis of the received signal may include determining the presence or absence and/or the concentration of albumin, for example, by comparing the received signal to a predetermined reference value. In addition, in one aspect of the present disclosure, the analysis result may be output to, for example, a display device connected to the sensor, another device connected via a network, or the like.

### EXAMPLES

Hereinafter, the present disclosure will be described more specifically with reference to examples, but the present disclosure is not limited to these examples.

### Synthesis Example 1: Synthesis of Europium Complex (Eu-1) Represented by the Following Formula

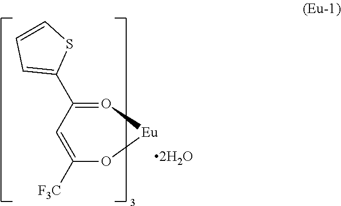

In a water/ethanol (2 ml/12 ml) mixed solvent, 0.18 g (4.5 mM) of sodium hydroxide is dissolved, 1 g (4.5 mM) of 4,4,4-trifluoro-1-(2-thienyl)-1,3-butanedione is added thereto, and after all the components are dissolved, the mixture obtained by dissolving 0.55 g (1.5 mM) of europium(III) chloride hexahydrate in 15 ml of water is added thereto, and stirred at room temperature. Precipitated crystals were filtered off, washed with water, and then dried under reduced pressure to obtain 0.23 g of a desired white europium complex Eu-1.

### Synthesis Example 2: Synthesis of Europium Complex (Eu-2) Represented by the Following Formula

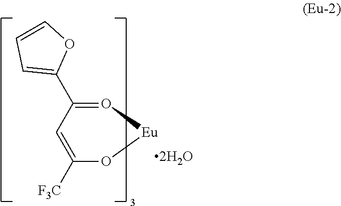

A europium complex Eu-2 was synthesized in the same manner as in Synthesis Example 1 except that 4,4,4-trifluoro-1-(2-furyl)-1,3-butanedione was used instead of 4,4,4-trifluoro-1-(2-thienyl)-1,3-butanedione.

### Synthesis Example 3: Synthesis of Europium Complex (Eu-3) Represented by the Following Formula

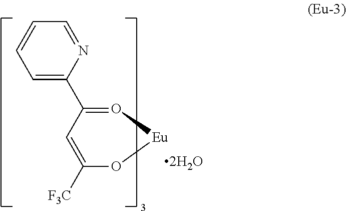

A europium complex Eu-3 was synthesized in the same manner as in Synthesis Example 1 except that 4,4,4-trifluoro-1-(2-pyridyl)-1,3-butanedione was used instead of 4,4,4-trifluoro-1-(2-thienyl)-1,3-butanedione.

### Synthesis Example 4: Synthesis of Europium Complex (Eu-4) Represented by the Following Formula

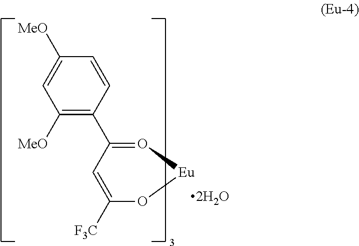

A europium complex Eu-4 was synthesized in the same manner as in Synthesis Example 1 except that 4,4,4-trifluoro-1-(2,4-dimethoxyphenyl)-1,3-butanedione was used instead of 4,4,4-trifluoro-1-(2-thienyl)-1,3-butanedione.

### Synthesis Example 5: Synthesis of Europium Complex (Eu-5) Represented by the Following Formula

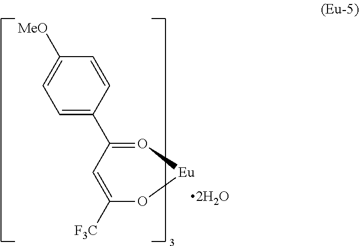

A europium complex Eu-5 was synthesized in the same manner as in Synthesis Example 1 except that 4,4,4-trifluoro-1-(4-methoxyphenyl)-1,3-butanedione was used instead of 4,4,4-trifluoro-1-(2-thienyl)-1,3-butanedione.

### Synthesis Example 6: Synthesis of Europium Complex (Eu-6) Represented by the Following Formula

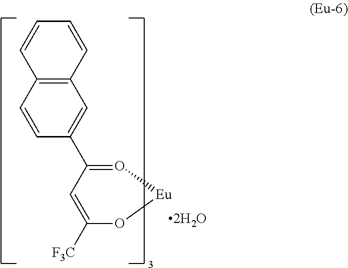

A europium complex Eu-6 was synthesized in the same manner as in Synthesis Example 1 except that 4,4,4-trifluoro-1-(2-naphthyl)-1,3-butanedione was used instead of 4,4,4-trifluoro-1-(2-thienyl)-1,3-butanedione.

### (Example 1) Reagent for Detecting HSA

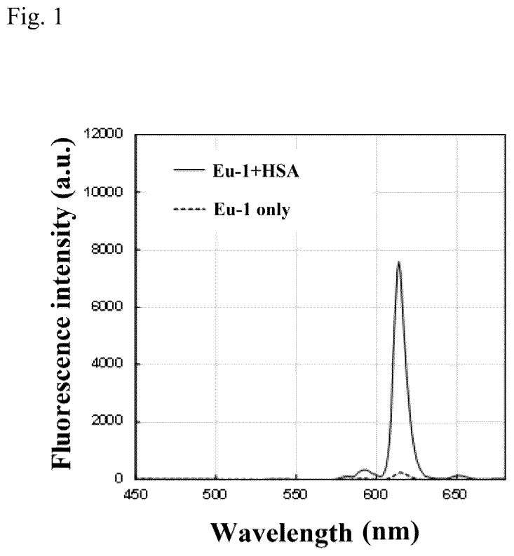

A dimethyl sulfoxide (DMSO) solution of the europium complex (Eu-1) obtained in Synthesis Example 1 was prepared (concentration: 0.5 mM), and an aqueous solution of albumin (HSA, derived from human serum, manufactured by FUJIFILM Wako Pure Chemical Corporation) was further prepared (concentration: 0.01 mg/ml). Then, 3 ml of an aqueous HSA solution was placed in a quartz cell, 30 μL of a DMSO solution of Eu-1 was added thereto, and the mixture was allowed to stand at room temperature for 2 minutes. Thereafter, the fluorescence spectrum with excitation light of 350 nm was measured. In addition, 30 μL of a DMSO solution of Eu-1 was added to 3 ml of water containing no HSA, and the fluorescence spectrum was measured in the same manner. The obtained fluorescence spectrum curve is shown in FIG. 1. The solid line represents the fluorescence spectrum of Eu-1+HSA, and the broken line represents the fluorescence spectrum of Eu-1 alone. The fluorescence intensity of Eu-1+HSA at a wavelength of 614 nm in the obtained fluorescence spectrum was 7,579. On the other hand, the fluorescence intensity of Eu-1 alone was 232. Therefore, it was found that the fluorescence emission intensity increased to about 33 times in the presence of HSA, indicating that HSA can be detected by fluorescence.

### (Example 2) Reagent for Detecting HSA

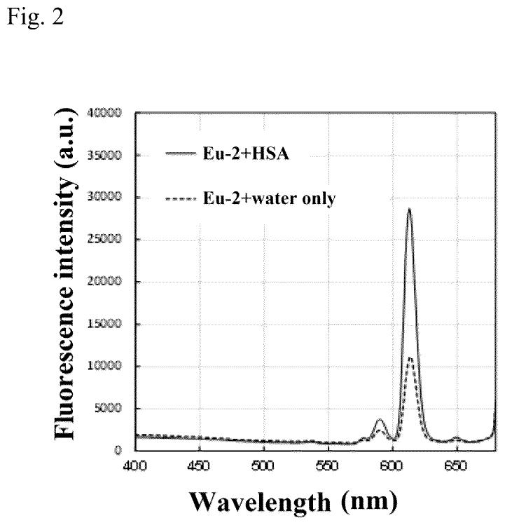

Evaluation was carried out in the same manner as in Example 1 except that the europium complex (Eu-2) obtained in Synthesis Example 2 was used instead of the europium complex (Eu-1) obtained in Synthesis Example 1. The obtained fluorescence spectrum curve is shown in FIG. 2. The solid line represents the fluorescence spectrum of Eu-2+HSA, and the broken line represents the fluorescence spectrum of Eu-2+water alone. It was found that the fluorescence intensity of Eu-2+HSA at a wavelength of 613 nm in the obtained fluorescence spectrum increased to 2.6 times the fluorescence intensity of Eu-2+water alone, indicating that HSA can be detected by fluorescence.

### (Example 3) Reagent for Detecting HSA

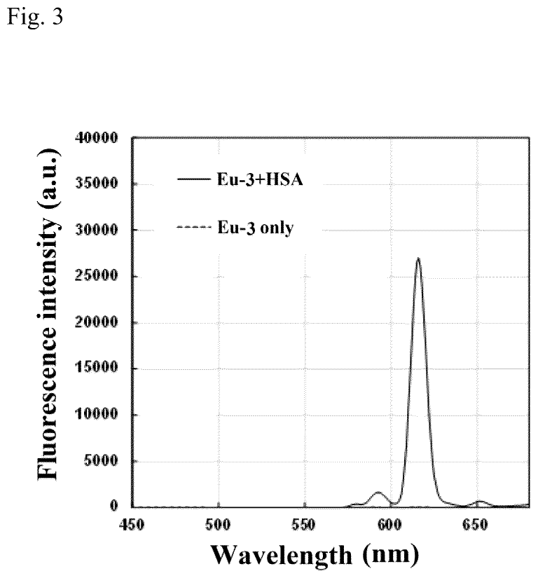

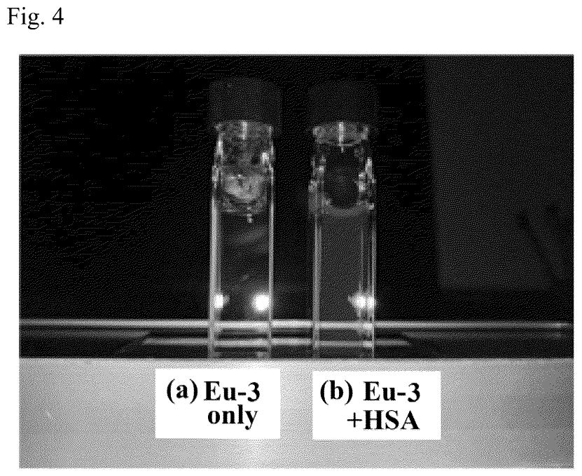

In the same manner as in Example 1, except that the europium complex (Eu-3) obtained in Synthesis Example 3 was used instead of the europium complex (Eu-1) obtained in Synthesis Example 1, the fluorescence spectrum was measured with excitation light of 365 nm. The obtained fluorescence spectrum curve is shown in FIG. 3. The solid line represents the fluorescence spectrum of Eu-3+HSA, and the broken line represents the fluorescence spectrum of Eu-3 alone. The fluorescence intensity of Eu-3+HSA at a wavelength of 616 nm in the obtained fluorescence spectrum was 27,013. On the other hand, the fluorescence intensity of Eu-3 alone was 62. Therefore, the fluorescence emission intensity increased to 436 times in the presence of HSA. A photograph of the quartz cells containing each solution obtained at this time in the light-emitting state excited by a UV lamp having a wavelength of 365 nm is shown in FIG. 4. In a solution (b) containing HSA, red emission peculiar to the europium complex was clearly confirmed in the image. On the other hand, in a solution (a) containing no HSA, it was confirmed from the image that the fluorescence was very weak. As described above, it was found that the presence of HSA can be confirmed also by the fluorescence image.

### (Example 4) Reagent for Detecting HSA

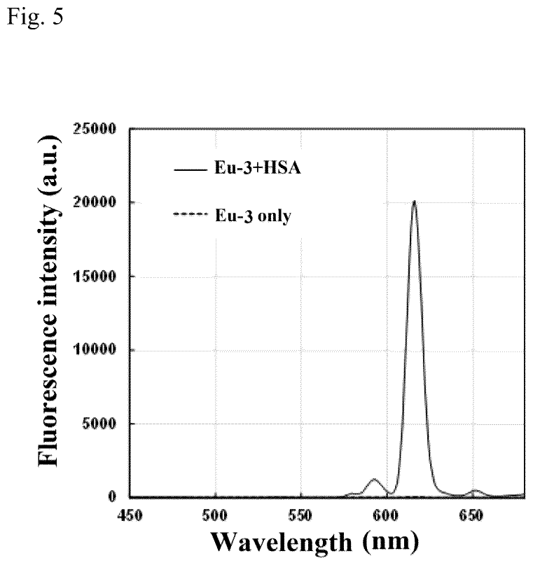

Evaluation was carried out in the same manner as in Example 3 except that the concentration of the aqueous solution of albumin (HSA, derived from human serum, manufactured by FUJIFILM Wako Pure Chemical Corporation) was changed from 0.01 mg/ml to 0.001 mg/ml. The fluorescence spectrum curve at excitation light of 365 nm is shown in FIG. 5. The solid line represents the fluorescence spectrum of Eu-3+HSA, and the broken line represents the fluorescence spectrum of Eu-3 alone. The fluorescence intensity of Eu-3+HSA at a wavelength of 616 nm in the obtained fluorescence spectrum was 20,138. On the other hand, the fluorescence intensity of Eu-3 alone was 62. As described above, it was found that even when the concentration is low, the fluorescence emission intensity increases 325 times due to the presence of HSA, and HSA can be detected by fluorescence.

### (Example 5) Reagent for Detecting HSA

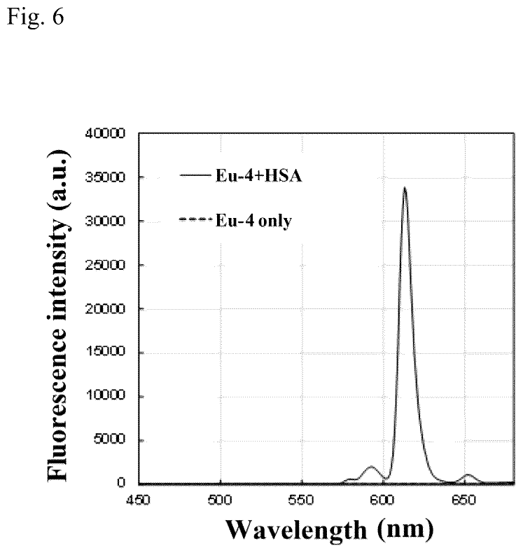

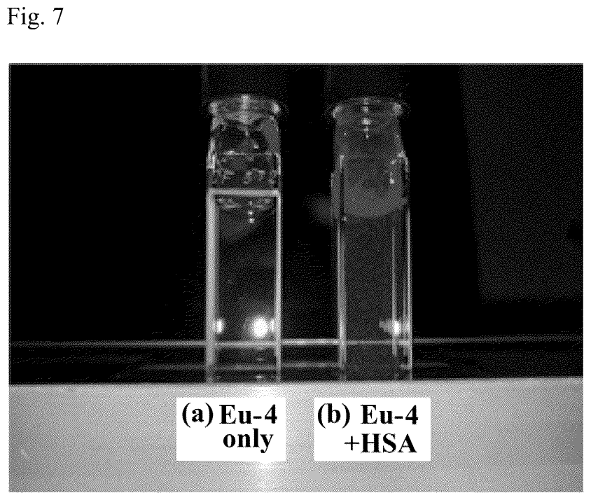

In the same manner as in Example 1, except that the europium complex (Eu-4) obtained in Synthesis Example 4 was used instead of the europium complex (Eu-1) obtained in Synthesis Example 1, the fluorescence spectrum was measured with excitation light of 365 nm. The obtained fluorescence spectrum curve is shown in FIG. 6. The solid line represents the fluorescence spectrum of Eu-4+HSA, and the broken line represents the fluorescence spectrum of Eu-4 alone. The fluorescence intensity of Eu-4+HSA at a wavelength of 613 nm in the obtained fluorescence spectrum was 33,841. On the other hand, the fluorescence intensity of Eu-4 alone was 34. Therefore, it was found that the fluorescence emission intensity increased to about 1,000 times in the presence of HSA, indicating that HSA can be detected by fluorescence. A photograph of the quartz cells containing each solution obtained at this time in the light-emitting state excited by a UV lamp having a wavelength of 365 nm is shown in FIG. 7. In a solution (b) containing HSA, red emission peculiar to the europium complex was clearly confirmed in the image. On the other hand, in a solution (a) containing no HSA, it was confirmed from the image that the fluorescence was very weak. As described above, it was found that the presence of HSA can be confirmed also by the fluorescence image.

### (Example 6) Reagent for Detecting HSA

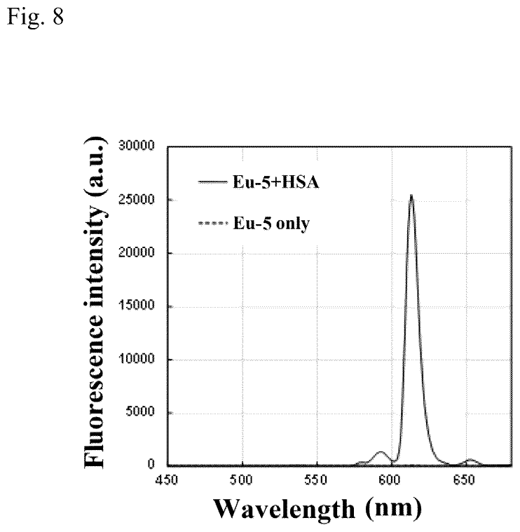

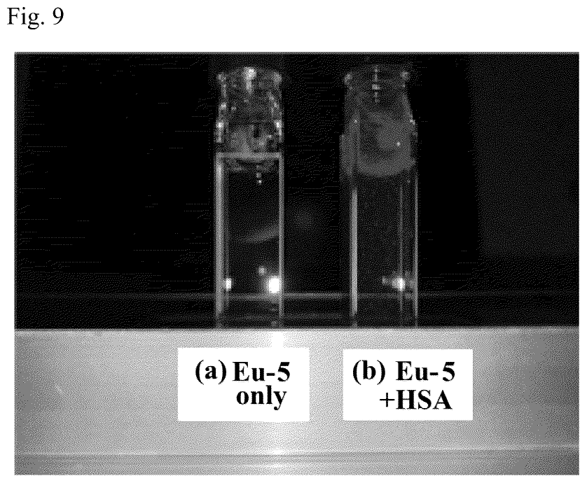

In the same manner as in Example 1, except that the europium complex (Eu-5) obtained in Synthesis Example 5 was used instead of the europium complex (Eu-1) obtained in Synthesis Example 1, the fluorescence spectrum was measured with excitation light of 350 nm. The obtained fluorescence spectrum curve is shown in FIG. 8. The solid line represents the fluorescence spectrum of Eu-5+HSA, and the broken line represents the fluorescence spectrum of Eu-5 alone. The fluorescence intensity of Eu-5+HSA at a wavelength of 613 nm in the obtained fluorescence spectrum was 25,504. On the other hand, the fluorescence intensity of Eu-5 alone was 58. Therefore, it was found that the fluorescence emission intensity increased to about 440 times in the presence of HSA, indicating that HSA can be detected by fluorescence. A photograph of the quartz cells containing each solution obtained at this time in light-emitting state excited by a UV lamp having a wavelength of 365 nm is shown in FIG. 9. In a solution (b) containing HSA, red emission peculiar to the europium complex was clearly confirmed in the image. On the other hand, in a solution (a) containing no HSA, it was confirmed from the image that the fluorescence was very weak. As described above, it was found that the presence of HSA can be confirmed also by the fluorescence image.

### (Example 7) Test Strip for Detecting HSA

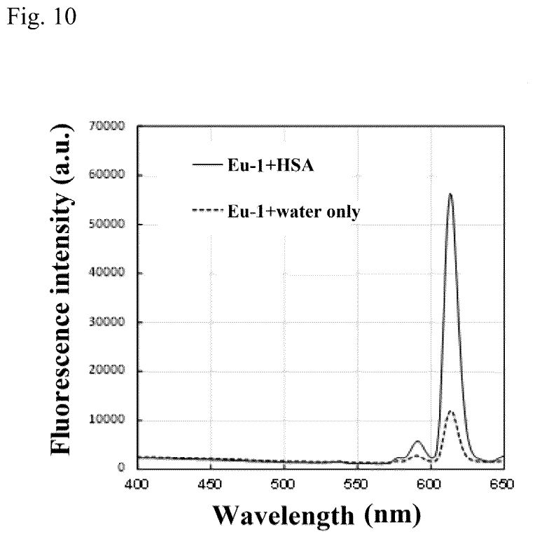

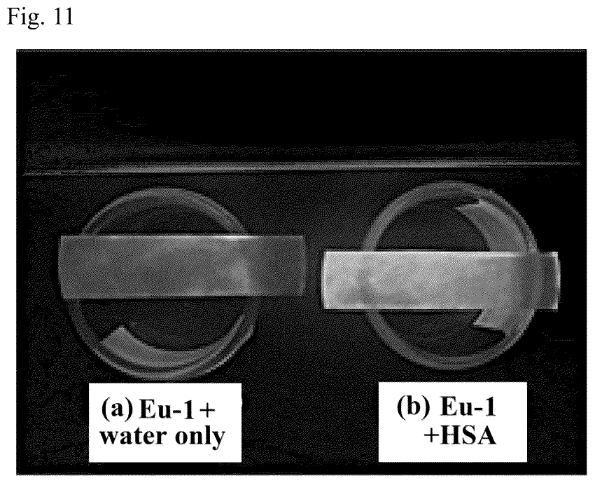

A mixed solution of the europium complex (Eu-1) obtained in Synthesis Example 1 in dimethyl sulfoxide (DMSO) and polyethylene glycol 200 (manufactured by Tokyo Chemical Industry Co., Ltd., volume ratio: 80/20) was prepared (concentration: 0.5 mM), and 0.2 ml of the solution was added dropwise to filter paper ((D: 40 mm) to impregnate the filter paper with the solution, and the filter paper was dried at 50° C. and cut with a width of 1 cm. In addition, an aqueous solution of albumin (HSA, derived from human serum, manufactured by FUJIFILM Wako Pure Chemical Corporation) was prepared (concentration: 0.1 mg/ml). Then, the filter paper impregnated with the europium complex was immersed for 30 seconds in a sample container (made of glass and having a capacity of 10 ml) containing 9 ml of an aqueous HSA solution, and the filter paper was taken out, and then allowed to stand at room temperature for 5 minutes. The fluorescence spectrum of the obtained filter paper with excitation light of 350 nm was measured. In addition, the fluorescence spectrum of the filter paper impregnated with the europium complex and immersed in 9 ml of water containing no HSA was also measured in the same manner. The obtained fluorescence spectrum curve is shown in FIG. 10. The solid line represents the fluorescence spectrum of Eu-1+HSA, and the broken line represents the fluorescence spectrum of Eu-1+water alone. It was found that the fluorescence intensity of Eu-1+HSA at a wavelength of 612 nm in the obtained fluorescence spectrum increased to about 3 times the fluorescence intensity of Eu-1+water alone, indicating that HSA can be detected by fluorescence. A photograph of the filter paper impregnated with the europium complex in light-emitting state excited by a UV lamp having a wavelength of 365 nm is shown in FIG. 11. In a filter paper (b) immersed in an aqueous solution containing HSA, red emission peculiar to the europium complex was clearly confirmed in the image. On the other hand, in a filter paper (a) immersed only in water without HSA, it was confirmed from the image that the fluorescence was very weak. As described above, it was found that the presence of HSA can be confirmed also by the fluorescence image.

### (Example 8) Test Strip for Detecting HSA

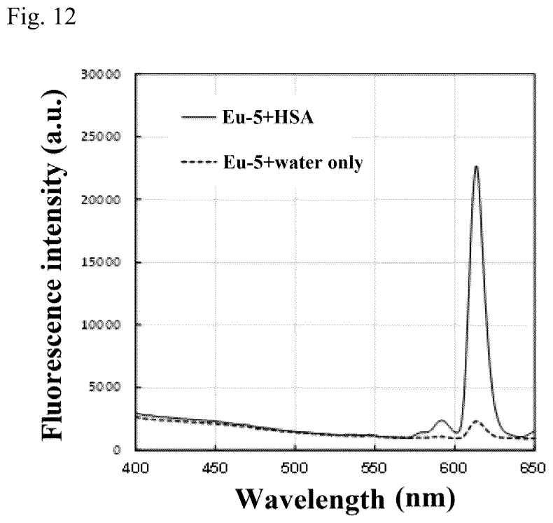

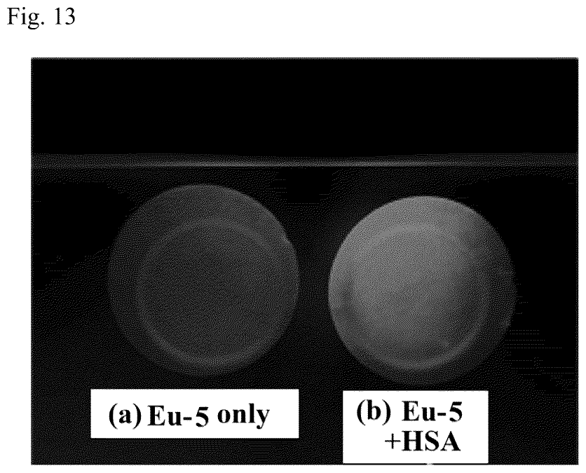

Evaluation was carried out in the same manner as in Example 7, except that the europium complex (Eu-5) obtained in Synthesis Example 5 was used instead of the europium complex (Eu-1) obtained in Synthesis Example 1, and a circular filter paper ((D: 40 mm) was used. The obtained fluorescence spectrum curve is shown in FIG. 12. The solid line represents the fluorescence spectrum of Eu-5+HSA, and the broken line represents the fluorescence spectrum of Eu-5+water alone. It was found that the fluorescence intensity of Eu-5+HSA at a wavelength of 614 nm in the obtained fluorescence spectrum increased to 10 times the fluorescence intensity of Eu-5+water alone, indicating that HSA can be detected by fluorescence. A photograph of the light-emitting state when the obtained test strip is excited by a UV lamp having a wavelength of 365 nm is shown in FIG. 13. In a test strip (b) immersed in a solution containing HSA, red emission peculiar to the europium complex was clearly confirmed in the image. On the other hand, in the test strip (a) immersed in a solution containing no HSA, it was confirmed from the image that the fluorescence was very weak. As described above, it was found that the presence of HSA can be confirmed also by the fluorescence image in the method using the test strip.

### (Example 9) Detection of HSA in Aqueous Solution Containing Various Ions (Sodium Ion, Potassium Ion, and Calcium Ion) and Nitrogen Compounds (Urea, Sodium Urate, and Creatinine) Present in Urine

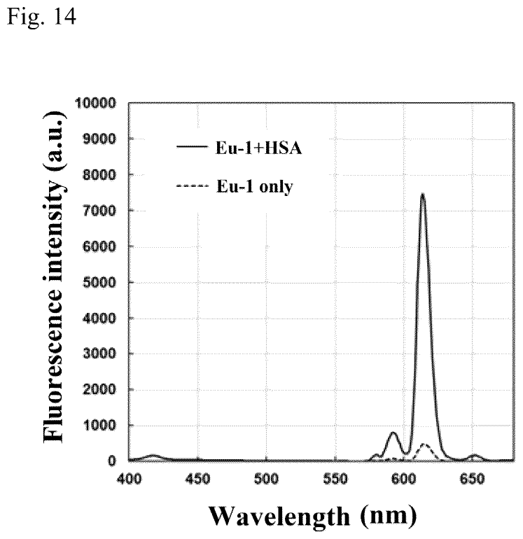

In 24.99 ml of water, 4.2 ml of a 1M aqueous sodium chloride solution, 0.12 ml of a 1M aqueous potassium chloride solution, 0.69 ml of a 0.1M aqueous calcium chloride solution, 6 mg of urea, 0.3 mg of creatinine, and 1.7 mg of sodium urate were dissolved to prepare an aqueous solution containing various ions and nitrogen compounds. Next, by using the present aqueous solution, an aqueous solution of albumin (HSA, derived from human serum, manufactured by FUJIFILM Wako Pure Chemical Corporation) was prepared (concentration: 0.01 mg/ml). Then, a dimethyl sulfoxide (DMSO) solution of the europium complex (Eu-1) obtained in Synthesis Example 1 was prepared (concentration: 0.5 mM). Then, 3 ml of an aqueous HSA solution was placed in a quartz cell, 30 μL of a DMSO solution of Eu-1 was added thereto, and the mixture was allowed to stand at room temperature for 2 minutes. Thereafter, the fluorescence spectrum with excitation light of 365 nm was measured. In addition, 30 μL of a DMSO solution of Eu-1 was added to 3 ml of the present aqueous solution containing no HSA, and the fluorescence spectrum was measured in the same manner. The obtained fluorescence spectrum curve is shown in FIG. 14. The solid line represents the fluorescence spectrum of Eu-1+HSA, and the broken line represents the fluorescence spectrum of Eu-1 alone. The fluorescence intensity of Eu-1+HSA at a wavelength of 615 nm in the obtained fluorescence spectrum was 7,269. On the other hand, the fluorescence intensity of Eu-1 alone was 485. As described above, it was found that the fluorescence emission intensity increased about 16 times in the presence of HSA, and urinary microalbumin could be detected by fluorescence.

### (Example 10) Detection of HSA in Aqueous Solution Containing Various Ions (Sodium Ion, Potassium Ion, and Calcium Ion) and Nitrogen Compounds (Urea, Sodium Urate, and Creatinine) Present in Urine

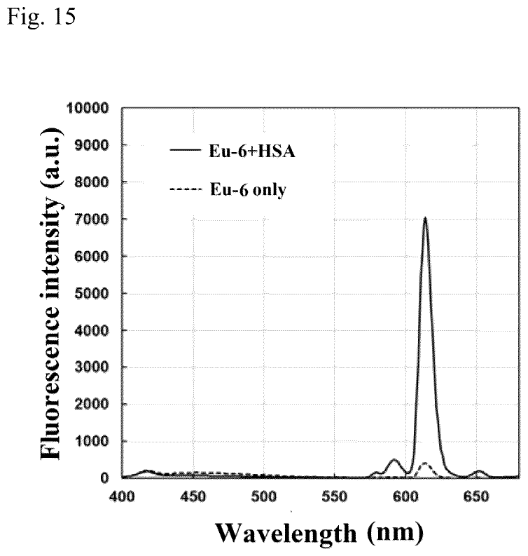

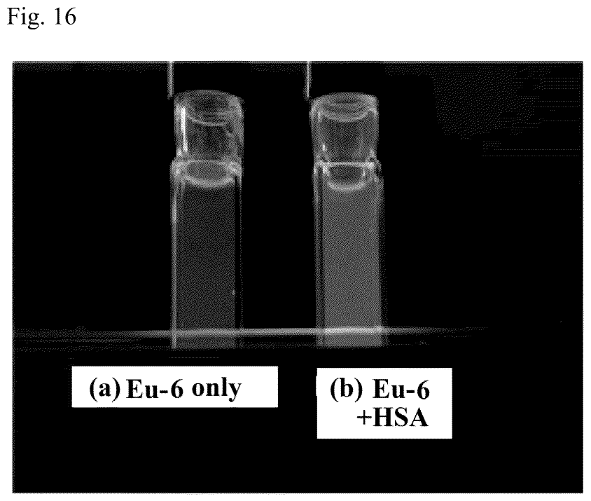

Evaluation was carried out in the same manner as in Example 9, except that the europium complex (Eu-6, concentration: 0.05 mM) obtained in Synthesis Example 6 was used instead of the europium complex (Eu-1) obtained in Synthesis Example 1. The obtained fluorescence spectrum curve is shown in FIG. 15. The solid line represents the fluorescence spectrum of Eu-6+HSA, and the broken line represents the fluorescence spectrum of Eu-6+water alone. It was found that the fluorescence intensity of Eu-6+HSA at a wavelength of 614 nm in the obtained fluorescence spectrum increased to 17 times the fluorescence intensity of Eu-6+water alone, indicating that HSA can be detected by fluorescence. A photograph of the quartz cells containing each solution obtained at this time in the light-emitting state excited by a UV lamp having a wavelength of 365 nm is shown in FIG. 16. In a solution (b) containing HSA, red emission peculiar to the europium complex was clearly confirmed in the image. On the other hand, in a solution (a) containing no HSA, it was confirmed from the image that the fluorescence was very weak. As described above, it was found that the presence of HSA can be confirmed also by the fluorescence image.

### (Example 11) Detection of HSA in Aqueous Solution Containing Various Ions (Sodium Ion, Potassium Ion, and Calcium Ion) and Nitrogen Compounds (Urea, Sodium Urate, and Creatinine) Present in Urine By Using Test Strip

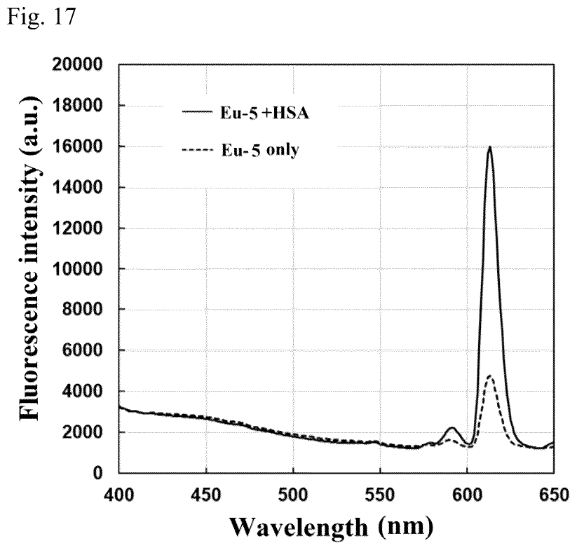

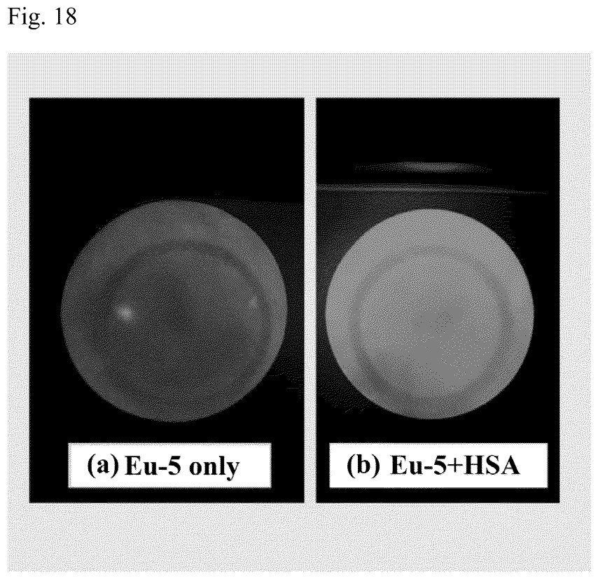

In 24.99 ml of water, 4.2 ml of a 1M aqueous sodium chloride solution, 0.12 ml of a 1M aqueous potassium chloride solution, 0.69 ml of a 0.1M aqueous calcium chloride solution, 6 mg of urea, 0.3 mg of creatinine, and 1.7 mg of sodium urate were dissolved to prepare an aqueous solution containing various ions and nitrogen compounds. Next, an aqueous solution of albumin (HSA, derived from human serum, manufactured by FUJIFILM Wako Pure Chemical Corporation) was prepared using the present aqueous solution (concentration of 0.1 mg/ml). Next, a mixed solution of the europium complex (Eu-5) obtained in Synthesis Example 5 in dimethyl sulfoxide (DMSO) and polyethylene glycol 200 (manufactured by Tokyo Chemical Industry Co., Ltd., volume ratio: 80/20) was prepared (concentration: 0.1 mM), and 0.2 ml of the solution was added dropwise to filter paper ((D: 40 mm) to impregnate the filter paper with the solution, and the filter paper was dried at 50° C. Next, the filter paper impregnated with the europium complex was immersed in a petri dish (made of glass) containing 4 ml of an aqueous HSA solution for 1 minute, and the filter paper was taken out, and then allowed to stand at room temperature for 3 minutes. The fluorescence spectrum of the obtained filter paper with excitation light of 365 nm was measured. In addition, the fluorescence spectrum of the filter paper impregnated with the europium complex and immersed in 4 ml of an aqueous solution containing no HSA was also measured in the same manner. The obtained fluorescence spectrum curve is shown in FIG. 17. The solid line represents the fluorescence spectrum of Eu-5+HSA, and the broken line represents the fluorescence spectrum of Eu-5 alone. In the obtained fluorescence spectrum, the fluorescence intensity of Eu-5+HSA at a wavelength of 613 nm was about 3.4 times the fluorescence intensity of Eu-5 alone. Therefore, it was found that the fluorescence emission intensity increased in the presence of HSA, indicating that HSA can be detected by fluorescence. A photograph of the filter paper impregnated with the europium complex in the light-emitting state excited by a UV lamp having a wavelength of 365 nm is shown in FIG. 18. In a filter paper (b) immersed in an aqueous solution containing HSA, red emission peculiar to the europium complex was clearly confirmed in the image. On the other hand, in a filter paper (a) immersed only in an aqueous solution without HSA, it was confirmed from the image that the fluorescence was very weak. As described above, it was found that even in the presence of various ions and nitrogen compounds, the presence of HSA can be confirmed by the fluorescence image of the test strip.

While the present disclosure has been particularly shown and described with reference to example embodiments and examples thereof, the present disclosure is not limited to these example embodiments and examples. It will be understood by those of ordinary skill in the art that various changes in form and details may be made therein without departing from the spirit and scope of the present disclosure as defined by the claims.

Some or all of the example embodiments described above may be described as in the following supplementary notes, but the disclosure of the present application is not limited to the following supplementary notes.

### (Supplementary Note 1)

A reagent for detecting albumin, the reagent comprising a europium complex represented by Formula (1):

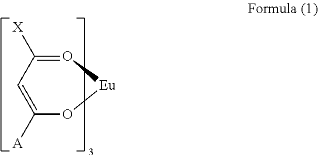

wherein X represents a substituted or unsubstituted aryl group or heterocyclic group, and A represents a substituted or unsubstituted alkyl group.

### (Supplementary Note 2)

The reagent according to supplementary note 1, wherein the substituted or unsubstituted aryl group has 6 to 18 carbon atoms.

### (Supplementary Note 3)

The reagent according to supplementary note 1 or 2, wherein the substituted or unsubstituted heterocyclic group has 2 to 30 carbon atoms.

### (Supplementary Note 4)

The reagent according to any one of preceding supplementary notes, wherein the substituted or unsubstituted alkyl group has 1 to 50 carbon atoms.

### (Supplementary Note 5)

The reagent according to any one of preceding supplementary notes, wherein X is a 2-thienyl group, a 2-furyl group, a 2-pyridyl group, a phenyl group, a 2-naphthyl group, a 4-hydroxyphenyl group, a 4-methoxyphenyl group, a 2,4-dimethoxyphenyl group, or a 4-fluorophenyl group.

### (Supplementary Note 6)

The reagent according to any one of preceding supplementary notes, wherein A is a trifluoromethyl group, a pentafluoroethyl group, or a heptafluoropropyl group.

### (Supplementary Note 7)

The reagent according to any one of preceding supplementary notes, further contains a solvent.

### (Supplementary Note 8)

The reagent according to supplementary note 7, wherein the solvent is dimethyl sulfoxide, methanol, ethanol, N,N-dimethylformamide, tetrahydrofuran, acetone, acetonitrile, or 1,4-dioxane.

### (Supplementary Note 9)

The reagent according to any one of preceding supplementary notes, further contains a glycol-based compound or glycerol.

### (Supplementary Note 10)

The reagent according to supplementary note 9, wherein the glycol-based compound is ethylene glycol, diethylene glycol, triethylene glycol, or polyethylene glycol.

### (Supplementary Note 11)

A method for detecting albumin in a sample using a europium complex represented by the following formula (1):

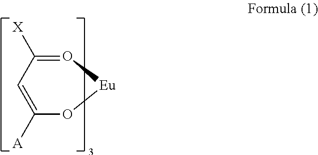

wherein X represents a substituted or unsubstituted aryl group or heterocyclic group, and A represents a substituted or unsubstituted alkyl group.

### (Supplementary Note 12)

The method according to supplementary note 11, wherein the substituted or unsubstituted aryl group has 6 to 18 carbon atoms.

### (Supplementary Note 13)

The method according to supplementary note 11 or 12, wherein the substituted or unsubstituted heterocyclic group has 2 to 30 carbon atoms.

### (Supplementary Note 14)

The method according to any one of supplementary notes 11 to 13, wherein the substituted or unsubstituted alkyl group has 1 to 50 carbon atoms.

### (Supplementary Note 15)

The method according to any one of supplementary notes 11 to 14, wherein X is a 2-thienyl group, a 2-furyl group, a 2-pyridyl group, a phenyl group, a 2-naphthyl group, a 4-hydroxyphenyl group, a 4-methoxyphenyl group, a 2,4-dimethoxyphenyl group, or a 4-fluorophenyl group.

### (Supplementary Note 16)

The method according to any one of supplementary notes 11 to 15, wherein A is a trifluoromethyl group, a pentafluoroethyl group, or a heptafluoropropyl group.

### (Supplementary Note 17)

The method for detecting albumin according to any one of supplementary notes 11 to 16, wherein the method utilizes a phenomenon in which fluorescence emission increases due to an interaction between the europium complex represented by the formula (1) and albumin.

### (Supplementary Note 18)

The method for detecting albumin according to any one of supplementary note 11 to 17, the method including:
(i) obtaining a mixture of a reagent containing the europium complex represented by the formula (1) and a sample;
(ii) irradiating the mixture with excitation light; and
(iii) detecting fluorescence emitted from the mixture.

### (Supplementary Note 19)

The method for detecting albumin according to supplementary note 18, wherein the excitation light has a wavelength of 200 to 500 nm.

### (Supplementary Note 20)

The method for detecting albumin according to any one of supplementary notes 11 to 19, wherein the sample is blood, plasma, serum, lymph, saliva, sweat, tears, or urine.

### (Supplementary Note 21)

The method for detecting albumin according to any one of supplementary notes 11 to 20, wherein the reagent containing the europium complex of the formula (1) is reacted with the sample in a solid medium containing the reagent.

### (Supplementary Note 22)

The method for detecting albumin according to supplementary note 21, wherein the solid medium is paper, glass, a resin, or a water-soluble polymer.

### (Supplementary Note 23)

A device for detecting albumin, the device including: an albumin capturing unit having the reagent according to any one of supplementary notes 1 to 10; and a detection unit for detecting that albumin has been captured in the capturing unit.

### (Supplementary Note 24)

The device for detecting albumin according to supplementary note 23, wherein the detection unit detects fluorescence emission of a reaction product of the europium complex represented by the formula (1) and albumin.

### (Supplementary Note 25)

A test strip for detecting albumin, the test strip containing the reagent according to any one of supplementary notes 1 to 10.

### (Supplementary Note 26)

A kit for detecting albumin, the kit containing the reagent according to any one of supplementary notes 1 to 10.

1. A reagent for detecting albumin, the reagent comprising a europium complex represented by Formula (1): 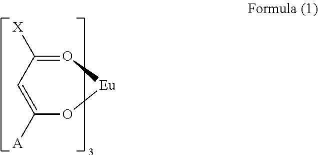
wherein X represents a substituted or unsubstituted aryl group or heterocyclic group, and A represents a substituted or unsubstituted alkyl group.

2. The reagent according to claim 1, wherein X is a 2-thienyl group, a 2-furyl group, a 2-pyridyl group, a phenyl group, a 2-naphthyl group, a 4-hydroxyphenyl group, a 4-methoxyphenyl group, a 2,4-dimethoxyphenyl group, or a 4-fluorophenyl group.

3. The reagent according to claim 1, wherein A is a trifluoromethyl group, a pentafluoroethyl group, or a heptafluoropropyl group.

4. The reagent according to claim 1, further comprises a solvent.

5. The reagent according to claim 1, further comprises a glycol-based compound or glycerol.

6. A method for detecting albumin in a sample using a europium complex represented by the following formula (1): 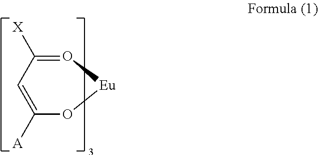
wherein X represents a substituted or unsubstituted aryl group or heterocyclic group, and A represents a substituted or unsubstituted alkyl group.

7. The method for detecting albumin according to claim 6, the method comprising:
(i) obtaining a mixture of a reagent containing the europium complex represented by the formula (1) and a sample;
(ii) irradiating the mixture with excitation light; and
(iii) detecting fluorescence emitted from the mixture.

8. A device for detecting albumin, the device comprising:
an albumin capturing unit having the reagent according to claim 1; and
a detection unit for detecting that albumin has been captured in the capturing unit.

9. The device for detecting albumin according to claim 8, wherein the detection unit detects fluorescence emission of a reaction product of the europium complex represented by the formula (1) and albumin.

10. A test strip for detecting albumin, the test strip comprising the reagent according to claim 1.

11. The reagent according to claim 1, wherein the substituted or unsubstituted aryl group has 6 to 18 carbon atoms, the substituted or unsubstituted heterocyclic group has 2 to 30 carbon atoms, and the substituted or unsubstituted alkyl group has 1 to 50 carbon atoms.

12. The reagent according to claim 4, wherein the solvent is dimethyl sulfoxide, methanol, ethanol, N,N-dimethylformamide, tetrahydrofuran, acetone, acetonitrile, or 1,4-dioxane.

13. The reagent according to claim 5, wherein the glycol-based compound is ethylene glycol, diethylene glycol, triethylene glycol, or polyethylene glycol.

14. The method according to claim 6, wherein X is a 2-thienyl group, a 2-furyl group, a 2-pyridyl group, a phenyl group, a 2-naphthyl group, a 4-hydroxyphenyl group, a 4-methoxyphenyl group, a 2,4-dimethoxyphenyl group, or a 4-fluorophenyl group.

15. The method according to claim 6, wherein A is a trifluoromethyl group, a pentafluoroethyl group, or a heptafluoropropyl group.

16. The method for detecting albumin according to claim 6, wherein the method utilizes a phenomenon in which fluorescence emission increases due to an interaction between the europium complex represented by the formula (1) and albumin.

17. The method for detecting albumin according to claim 7, wherein the excitation light has a wavelength of 200 to 500 nm.

18. The method for detecting albumin according to claim 6, wherein the sample is blood, plasma, serum, lymph, saliva, sweat, tears, or urine.

19. The method for detecting albumin according to claim 6, wherein the reagent comprising the europium complex of the formula (1) is reacted with the sample in a solid medium comprising the reagent.

20. The method for detecting albumin according to claim 19, in which the solid medium is paper, glass, a resin, or a water-soluble polymer.
